(19) United States
(12) Patent Application Publication
(10) Pub. No.: US 2019/0374191 A1
(43) Pub. Date: Dec. 12, 2019

(54) INFORMATION-PROCESSING DEVICE, PROGRAM, TABLE AND RADIATION IMAGING APPARATUS

(71) Applicant: SHIMADZU CORPORATION, Kyoto-shi (JP)

(72) Inventors: Koki Yoshida, Kyoto-shi (JP); Hiroshi Inoue, Kyoto-shi (JP); Daisuke Murakami, Kyoto-shi (JP); Dai Hirose, Kyoto-shi (JP)

(73) Assignee: SHIMADZU CORPORATION, Kyoto-shi (JP)

(21) Appl. No.: 16/002,726

(22) Filed: Jun. 7, 2018

(51) Int. Cl. A61B 6/00; A61G 13/04; A61G 13/00; A61B 6/04

(52) U.S. Cl. CPC A61B 6/547; A61G 13/04; A61G 13/0018; A61G 2203/10; A61B 6/0457; A61G 2203/42; A61B 6/0407

(57) ABSTRACT

An examination table system provides a table that is securely moved with no loss of visibility on a radiograph due to bending or tilting. The table is equipped with an accelerometer and is rotatable from a horizontal position during use while supporting a patient to be imaged. The table may be manually moved. The system allows easy table movement compensation that incorporates the projection of the accelerometer and a table gradient while obtaining a clear radiograph image.

### CROSS REFERENCE TO RELATED APPLICATIONS

This application relates to, but does not claim priority from, JP 2015-234911 filed Dec. 1, 2015, the entire contents of which are incorporated herein by reference.

### FIGURE SELECTED FOR PUBLICATION

FIG. 8

### BACKGROUND OF THE INVENTION

### Field of the Invention

The present invention relates to an examination (operation) table system that an operator operates manually for movement thereof and on which a subject is loaded and particularly relates to each aspect of an information processing device, a program and a table applied to a radiation imaging apparatus.

### Description of the Related Art

FIG. 17 is illustrating one example of radiation imaging apparatuses. Such radiation imaging apparatus comprises the examination table 50 (exam table), a table 52 on which a subject is loaded, a pedestal 51 that supports the table 52, and a radiation source 53 and a detector 54 relative to radiation imaging. A C-arm 55 supports the radiation source 53 and the detector 54, and a support column 57 supports the C-arm 55.

Such radiation imaging apparatus is operative to change the positioning of the subject relative to the C-arm 55. The subject is moved relative to the C-arm 55 so that the region of the subject to be imaged is changed. The examination table 50 is operative to achieve such positioning change relative to the subject. Specifically, referring to FIG. 18, the table 52 of the examination table 50 is operative to be movable relative to the pedestal 51. The table 52 is moved manually by an operator standing by the table 52. Specifically, referring to FIG. 19, a lever, which the operator grips, is mounted on the side of the table 52, and the operator adds the force to the table 52 through such a lever. Referring to FIG. 18, the operator slides the table 52 horizontally.

In addition, the pedestal 51 support the table 52 to be freely rotatable. Specifically, referring to FIG. 20, the table 52 is rotatable and movable around the axis parallel to the body axis of the subject. A rotation mechanism inside the pedestal 51 achieves such a rotation-movement but not manually by the operator. An operation panel is installed besides the table 52 and the operator control rotation of the table 52 through the operation panel.

It is dangerous that the table 52 automatically slips down when the table 52 is intended to be moved in the body axis direction of the subject while the table 52 is tilting. Therefore, the examination table 50 is under safety measures to prevent such an incident. Specifically, referring to FIG. 21, the examination table 50 is equipped with an angle sensor that detects the gradient-angle of the table 52. Such an angle sensor is installed to the rotation mechanism of the table 52 and the gradient-angle of the table 52 is monitored based on the rotation axis.

Given the table 52 rotates from the horizontal position, the angle sensor detects that the table 52 transits from the horizontal position to the tilting position due to such a rotation. The examination table 50 prohibits the movement of the table 52 along the body axis of the subject so that the table 52 does not automatically slip down from such a position in which the table 52 is not horizontal. Therefore, the table 52 is now in locking position, the table 52 does not move in the body axis direction of the subject even when the operator adds the force onto the lever. Such a locking means that the condition prohibits the operator to move the table manually, but rotation thereof is not locked. Even when in the locking position, the operator is allowed to rotate the table 52 through the operation panel.

Referring to FIG. 22, given the table 52 rotates from the horizontal position, the angle sensor detects that the table 52 transits from the horizontal position to the tilting position due to such a rotation. The examination table 50 receives the directive for unlocking the table 52 from the operator. Referring to FIG. 22, once the operator provides the directive for unlocking the table 52 through the operation panel, the table 52 is unlocked so that the operator manually moves the table 52 in the body-axis direction of the subject.

It is known that such a conventional protocol is problematic. Specifically, referring to FIG. 23, the table 52 bends due to the weight of the subject. Such an incident likely takes place when the body weight of the subject is heavy. The bending level of the table 52 depends on the body weight of the subject.

Given the table 52 bends, the table 52 slightly tilts in addition. The angle sensor appurtenant to the pedestal 51 is incapable of detecting bending of the table 52. The reason is that such an angle sensor just monitors the gradient-angle of the table 52 from the rotation axis. Accordingly, even when the table 52 slightly tilts due to the bending of the table 52, the angle sensor determines the gradient of the table 52 without counting the bending of the table 52. Given the operator provides the directive for unlocking the table 52 while the angle sensor is detecting that the table 52 is horizontal, locking of the table 52 is canceled in the state in which the table 52 is bending and slightly tilting. Subsequently, the operator must manually enable the movement of the table 52 in the body-axis direction of the subject.

Such a bending of the table 52 is subtle. Therefore, the table 52 never automatically slips down under such a degree of the gradient. Regardless, the manual operation for operator is more difficult due to the gradient of the table 52. Specifically, referring to FIG. 24, the tilted table 52 is always forced to slip down. Referring to FIG. 24, such a force forces the table 52 to move in the direction indicated by the rightward arrow. Under such a state, when the operator gripping the lever tries to move the table 52 in the direction indicated by the leftward arrow, the force generated with slipping down resists such an action. Accordingly, the operator feels all the heavier to move the table 52.

Therefore, according to the conventional aspects, referring to FIG. 25, an accelerometer is equipped to detect the bending of the table 52. When the accelerometer detects the gradient of the table 52, the operator is not allowed to cancel the locking of the table 52. Referring to FIG. 26, firstly, the accelerometer detects that the table 52 is horizontal and then, the operator manually enables the movement of the table 52. The degree of the bending of the table 52 varies depending on the body weight of the subject. Accordingly, whether the table 52 is horizontal is unknown until the operator actually measures the condition of the table using the accelerometer (e.g., refer to Patent Document 1, Patent Document 2, Patent Document 3, Patent Document 4).

### RELATED PRIOR ART DOCUMENTS

### Patent Document

### Patent Document 1: JP Patent Published 2005-124615 A

### Patent Document 2: US Patent Published 2009-144902 A1

### Patent Document 3: US Patent Published 2007-200396 A1

### Patent Document 4: US Patent Published 2011-296613 A1

### ASPECTS AND SUMMARY OF THE INVENTION

### Objects to be Solved

However, the following problems remains in the conventional system.

Specifically, according to the conventional aspect, the visibility (visual recognition) is affected relative to X-ray imaging.

The accelerometer is mandatory to be mounted on the table 52. Otherwise the bending of the table 52 is not measurable. As a result, referring to FIG. 27, the radiograph obtained by the radiation imaging incorporates the projection of the accelerometer. Specifically, the visibility of the radiograph is impaired due to the equipped accelerometer.

To solve the above problems considering such a circumstance, one purpose of the present invention is to provide an examination table for an examination table system, which is operative while securing the operability of the table without effecting the visibility of a radiograph (radiation image).

### Means for Solving the Problem

The present invention is related to the following structures to solve the above objects.

Specifically, an examination table system applicable for a radiation imaging apparatus, according to the present invention, comprises an information processing device, a table through which X-ray is transmittable and on which a subject is loaded, a pedestal that supports the table of which an operator manually enables a moving along a body axis direction of the subject, an input device that the operator inputs a directive, a rotation mechanism that rotates the table around a rotation axis, a tilt sensor that detects a gradient of the table by monitoring an action of the rotation mechanism, a rotation control circuit that controls the rotation mechanism, and a movement locking mechanism that locks a movement of the table so that the table is prevented from the movement in the body-axis direction, wherein the information processing device further comprises a control circuit that controls the table on which the subject subjected to radiation imaging is loaded, (A) a table rotation circuit that rotates the table in accordance with the directive relative to rotation from the operator through the input device, (B1) a correction circuit that corrects a relationship between an output of the tilt sensor and a tilt angle of the table so that the output of the tilt sensor denotes that the table is horizontal upon being provided the directive of a correction-execution when the operator provides the directive of the correction-execution through the input device, (C) a movement-locking control circuit that cancels the movement-locking of the table when the tilt sensor outputs data denoting that the table is horizontal given the operator provides the directive for the movement-unlocking through the input device.

### Action and Effect

The inventor sets forth three specific approaches to solve the above objects in the present specification. According to the first aspect of the approach, the imaging processing device comprises the movement-locking control circuit that cancels the movement-locking of the table even though the table is not horizontal when the operator provides a directive for adjusting the gradient-angle detection through the input device, and the correction circuit that corrects the output of the tilt sensor so that the table is deemed horizontal at the time when the operator provides the directive for the correction-execution through the input device.

According to the first aspect thereof, it is not mandatory to actually measure the bending degree, so that the accelerometer is no longer mandatory to be equipped. The bending of the table varies depending on the body weight of the subject, so that the operator must actually manually conduct a measurement to know the degree of gradient of the table due to the bending. According to the conventional aspects, the table is equipped with the accelerometer, whereby the gradient of the table, which is actually measured, is canceled by rotating the table so that the table is maintained as a horizontal position, according to the aspect of the present invention, the situation, in which the operator slides the table while changing the angle of the table and realizes that the table is easily slidable, is registered as that the table is horizontal (flat). In such a way, as long as the operator adjusts directly the table to be horizontal, the operator does not have to know the gradient-angle of the table due to the bending using the equipment. According to the aspect of the present invention, the examination table that allows the operator to slide manually and easily is provided, whereby the table is maintained as a horizontal position without equipping the table with the accelerometer that disturbs the radiation imaging.

In addition, the examination table system, according to the present invention, comprises the information processing device, the table through which X-ray is transmittable and on which the subject is loaded, the pedestal that supports the table of which an operator manually enables a moving along a body axis direction of the subject, the input device that the operator inputs the directive, the rotation mechanism that rotates the table around a rotation axis, the tilt sensor that detects the gradient of the table by monitoring the action of the rotation mechanism, the rotation control circuit that controls the rotation mechanism, the movement locking mechanism that locks the movement of the table so that the table is prevented from the movement in the body-axis direction, wherein the information processing device further comprises a control circuit that controls the table on which the subject subjected to the radiation imaging is loaded, (m) a memory storage element that stores a table of data (hereinafter data-table) denoting a relationship between a bending-angle that is an angle indicating the degree of the gradient of the table due to the bending of the table and a position of the subject to be loaded on the table and the body weight of the subject loaded on the table, (B2) the correction circuit that corrects the relationship between an output of the tilt sensor and the gradient-angle of the table so that the updated state, in which the table rotates as much as the bending angle in the opposing direction against the bending of the table while the tilt sensor is detecting the horizontal state, is treated as a horizontal state by reading out the gradient-angle corresponding to the position of the subject when the body weight of the subject and the position thereof are input, and (C) the movement locking control circuit that cancels the movement-locking of the table when the tilt sensor outputs data denoting that the table is horizontal given the operator provides the directive for the movement-unlocking through the input device.

### Action and Effect

The inventor sets forth three specific approaches to solve the above objects in the present specification, According to the second aspect of the present invention, the memory storage element that stores the data-table denoting the relationship between the bending-angle that is the angle indicating the degree of the gradient of the table due to the bending of the table and the position of the subject to be loaded on the table and the body weight of the subject loaded on the table, and the correction circuit that corrects the output of the tilt sensor so that the updated state, in which the table rotates as much as the bending angle in the opposing direction against the bending of the table while the tilt sensor is detecting the horizontal state, is treated as a horizontal state by reading out the gradient-angle corresponding to the position of the subject when the body weight of the subject and the position thereof are input.

According to the second aspect thereof, it is not mandatory to actually measure the bending degree due to the bending, so that the accelerometer is no longer mandatory to be mounted. The bending of the table varies depending on the body weight of the subject and the position thereof, so that the operator must actually conduct such a measurement to know the degree of gradient of the table due to the bending. According to the conventional aspects, the table is equipped with the accelerometer, whereby the gradient of the table, which is actually measured, is canceled by rotating the table so that the table is maintained as a horizontal position, according to the above aspect of the present invention, the inventor notices a relationship existing between the bending degree and the body weight of the subject to be loaded on the table. The inventor of the present invention realizes that the degree of the bending of the table is comprehensible if the body weight of the subject is known. Specifically, the actual measurement of the level of the bending of the table is no longer mandatory. According to the aspect of the present invention, the examination table that allows the operator to slide manually and easily is provided, whereby the table is maintained as a horizontal position without equipping the table with the accelerometer that disturbs the radiation imaging.

In addition, the examination table system, according to the present invention, comprises the information processing device, the table through which X-ray is transmittable and on which the subject is loaded, the pedestal that supports the table of which an operator manually enables a moving along a body axis direction of the subject, the input device that the operator inputs a directive, the rotation mechanism that rotates the table around the rotation axis of the table, the tilt sensor that detects the gradient of the table by monitoring the action of the rotation mechanism, the rotation control circuit that controls the rotation mechanism, the movement locking mechanism that locks the movement of the table so that the table is prevented from the movement in the body-axis direction, wherein the information processing device further comprises a control circuit that controls the table on which the subject subjected to the radiation imaging is loaded, (B3) a correction circuit that corrects the relationship between an output of the tilt sensor and a gradient-angle of said table so that an updated state, in which the table rotates as much as a predetermined angle in an opposing direction against bending of the table while the tilt sensor is detecting a horizontal state, is treated as a horizontal state when the operator provides a directive to execute correction through the input device, and (C) the movement-locking control circuit that cancels the movement-locking of the table when the tilt sensor outputs data denoting that the table is horizontal given the operator provides the directive for canceling the movement-locking through the input device.

### Action and Effect

The inventor sets forth three specific approaches to solve the above objects in the present specification, According to the third aspect of the present invention, the correction circuit that corrects the output of the tilt sensor so that the updated state, in which the table rotates as much as a predetermined angle in the opposing direction against the bending of the table while the tilt sensor is detecting the horizontal state, is treated as a horizontal state when the operator provides the directive to execute the correction through the input device.

According to the third aspect thereof, it is not mandatory to actually measure the angle indicating the gradient degree of the table due to the bending, so that the accelerometer is no longer mandatory to be mounted. The bending of the table varies depending on the body weight of the subject, so that the operator must actually manually conduct a measurement to know the degree of gradient of the table due to the bending. According to the conventional aspects, the table is equipped with the accelerometer, whereby the gradient of the table, which is actually measured, is canceled by rotating the table, so that the table is maintained as a horizontal position. On the other hand, according to such a conventional aspect, the incorporation of the image of the accelerometer, which is set forth referring to FIG. 27, is not recognized. In contrast, according the above aspect of the present invention, the gradient due to the bending of the table is approximately predicted and then the operator maintains the table as a horizontal state by rotating the table as predicted. According such a aspect, an accuracy of the horizontal level of the table to be maintained is certainly lesser than the case in which the gradient of the bending table is actually measured. Regardless, according to the aspect of the above aspect of the present invention, the larger advantage, in which no image of the accelerometer is incorporated, is attained by incorporating no image of the accelerometer. According to the aspect of the present invention, the examination table that allows the operator to slide manually and easily is provided, whereby the table is maintained as a horizontal position without equipping the table with the accelerometer that disturbs the radiation imaging.

In addition, it is further preferable that the information processing device further comprises a formatting (initializing) circuit that defaults the relationship between the corrected output of the tilt sensor and the gradient of the table to the initial state.

### Action and Effect

The above aspect illustrates further specifically the examination table of the present invention. According to such an initializing circuit, the correction of the relationship between the corrected output of the tilt sensor and the gradient of the table is canceled to be in the default every time when the imaging of the subject ends, so that the correction action can be repeated every subject.

In addition, according to the examination table of the third aspect, it is further preferable that the correction circuit is operative using the predetermined angle that the operator selects through the input device from a plurality of the predetermined angles.

### Action and Effect

The above aspect illustrates further specifically the examination table of the present invention. According to the above aspect, the operator adjusts the angle relative to the correction and looks for the more appropriate gradient-angle for the movement of the table.

In addition, according to the information processing device, it is further preferable that the rotation control circuit controls the rotation mechanism to maintain the table as a horizontal state in accordance with the output of the tilt sensor.

### Action and Effect

The above aspect illustrates further specifically the examination table of the present invention. According to the technology aspect of the present invention, the operator can make the table immediately horizontal (flat).

### Effect of the Invention

According to the conventional aspects, the table is equipped with the accelerometer, whereby the gradient of the table, which is actually measured, is canceled by rotating the table so that the table is maintained as a horizontal position. Regardless, according to the aspect of the conventional aspect referring to FIG. 26, it is problematic that the radiograph incorporates the projection of the accelerometer. According to the present invention, the operator maintains the table as a horizontal state without the accelerometer. Therefore, the operator never feels heavy when manually moving the table and in addition, can obtain a clear image by using such an examination table.

The above and other aspects, features and advantages of the present invention will become apparent from the following description read in conjunction with the accompanying drawings, in which like reference numerals designate the same elements.

### BRIEF DESCRIPTION OF THE DRAWINGS

FIG. 1 is a functional block diagram illustrating an entire structure of an examination table according to the aspect of Embodiment 1.

FIGS. 2A, 2B are schematic views illustrating locking of a table according to the aspect of Embodiment 1.

FIGS. 3A, 3B are schematic views illustrating locking of the table according to the aspect of Embodiment 1.

FIG. 4 is a functional block diagram illustrating unlocking of the table according to the aspect of Embodiment 1.

FIGS. 5A, 5B are schematic views illustrating bending of the table according to the aspect of Embodiment 1.

FIG. 6 is a flow-chart illustrating a brief operation relative to horizontal resetting according to the aspect of Embodiment 1.

FIGS. 7A, 7B are schematic diagrams illustrating an angle adjustment operation according to the aspect of Embodiment 1.

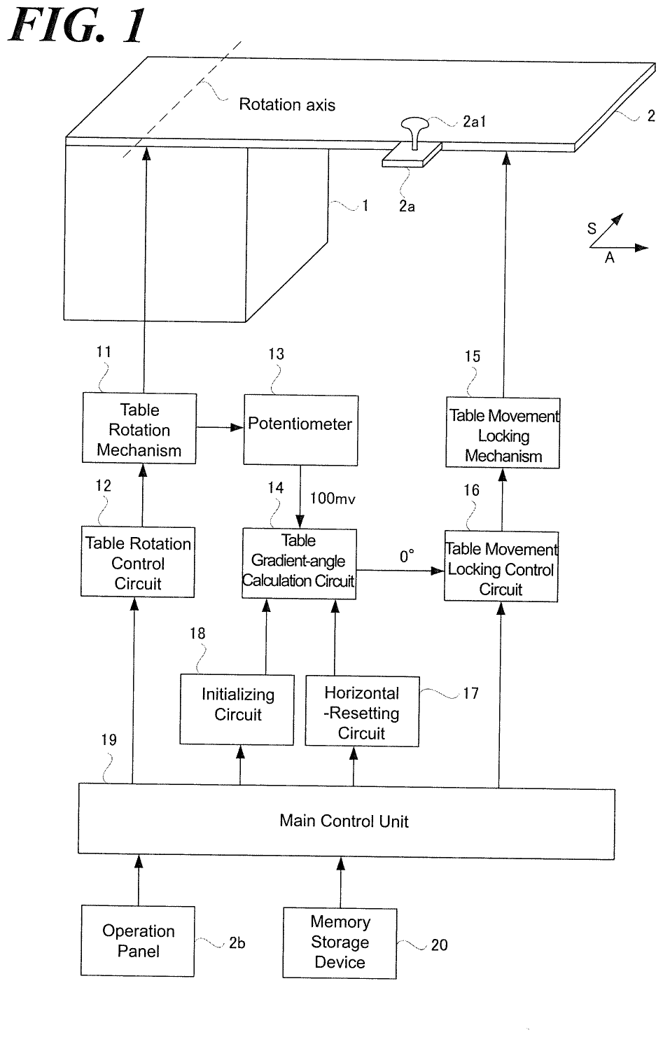

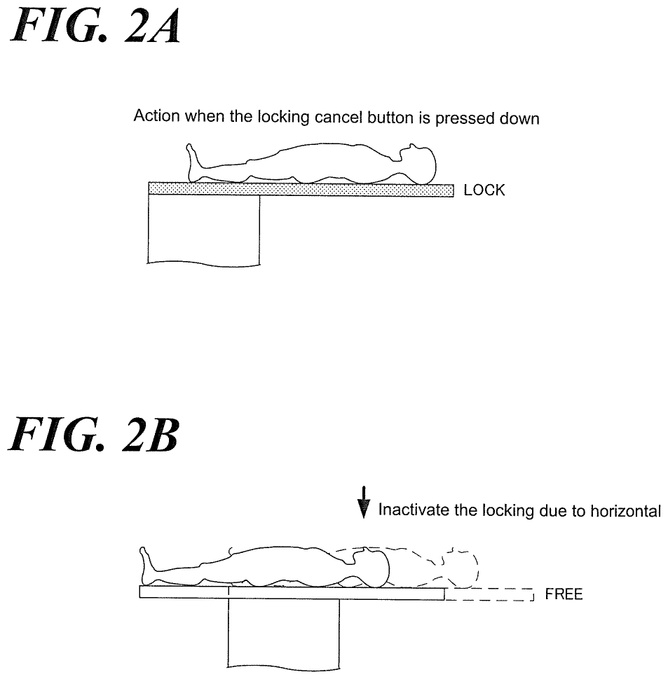

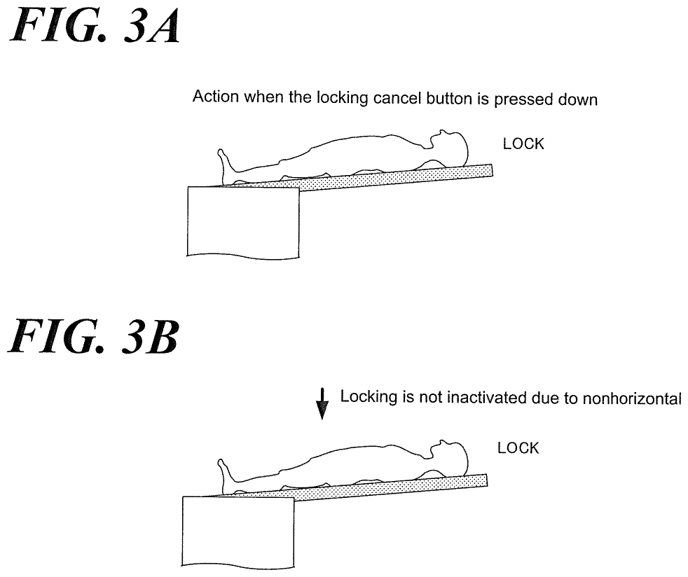

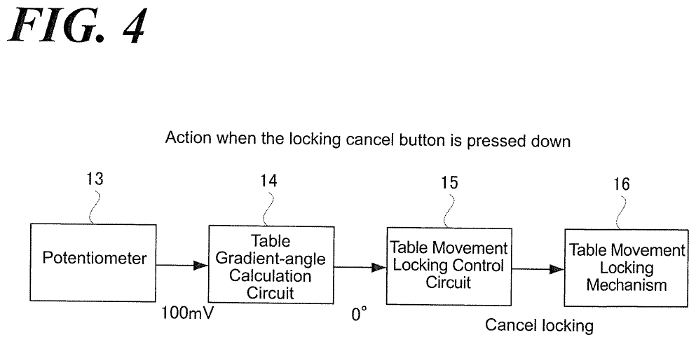

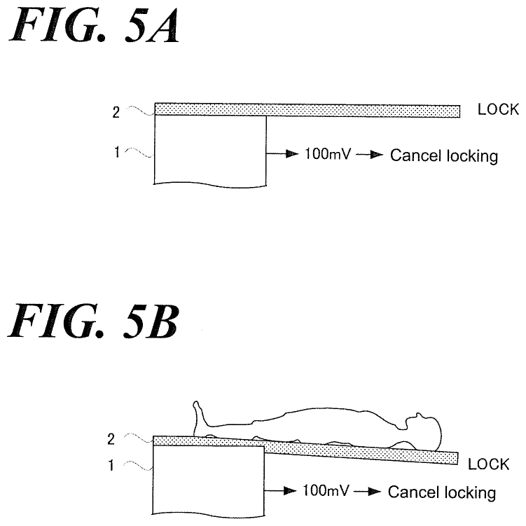

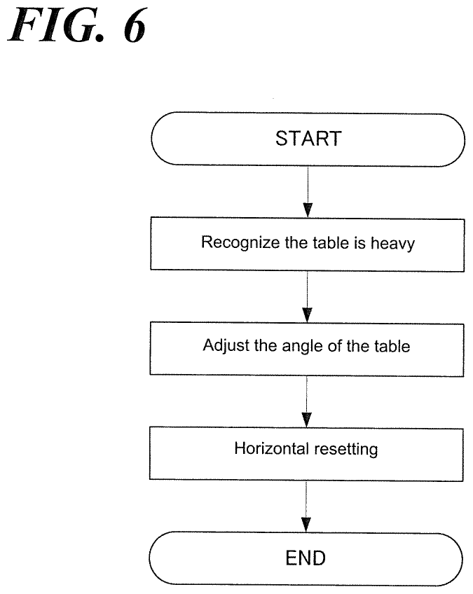

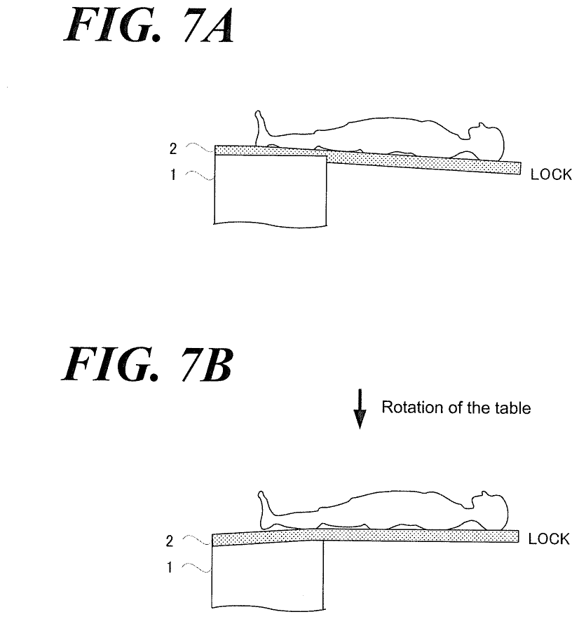

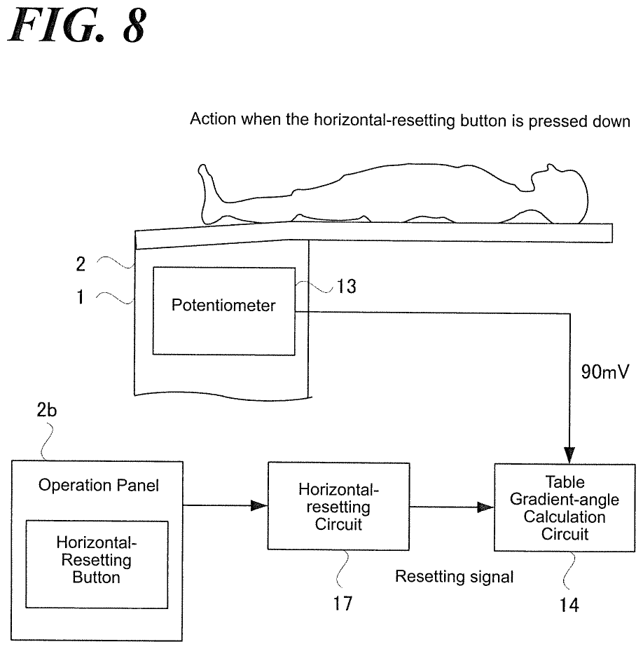

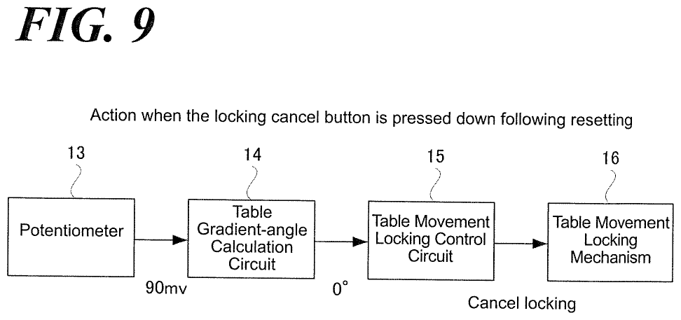

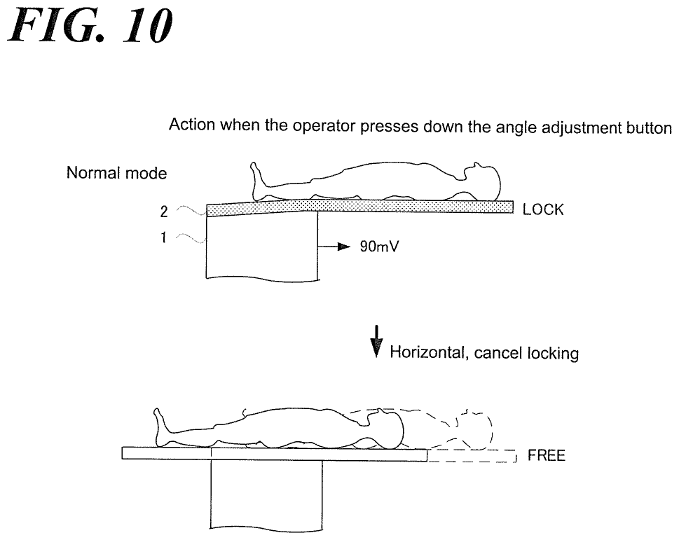

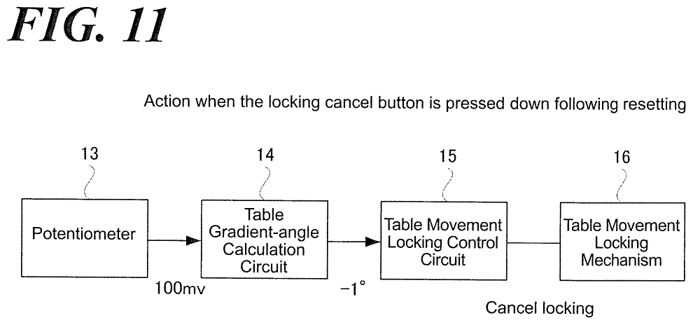

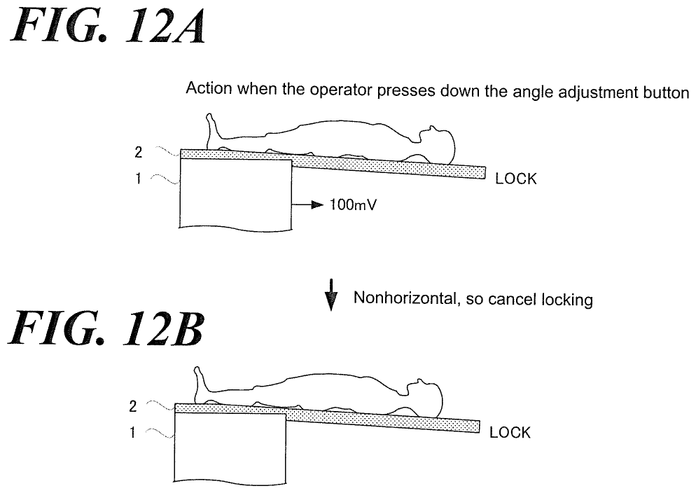

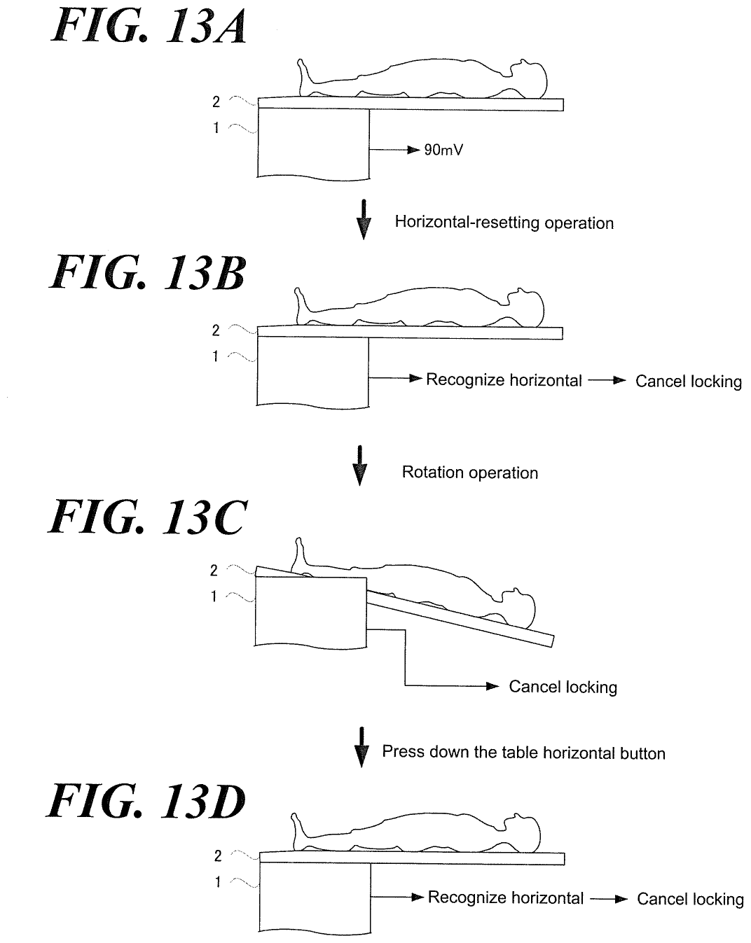

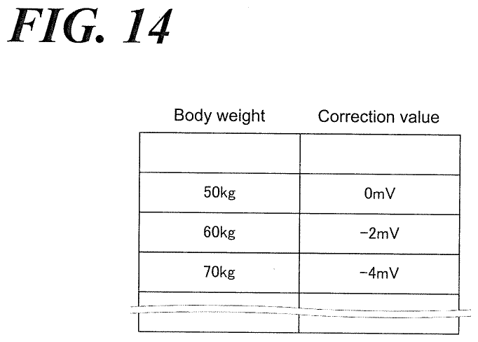

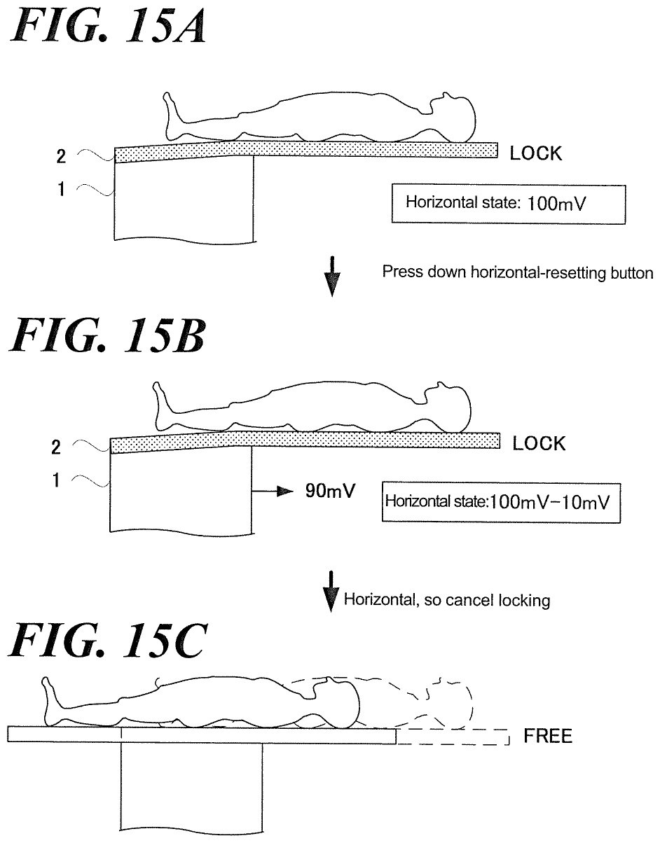

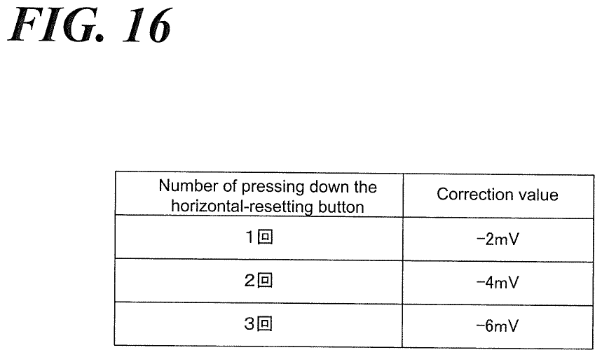

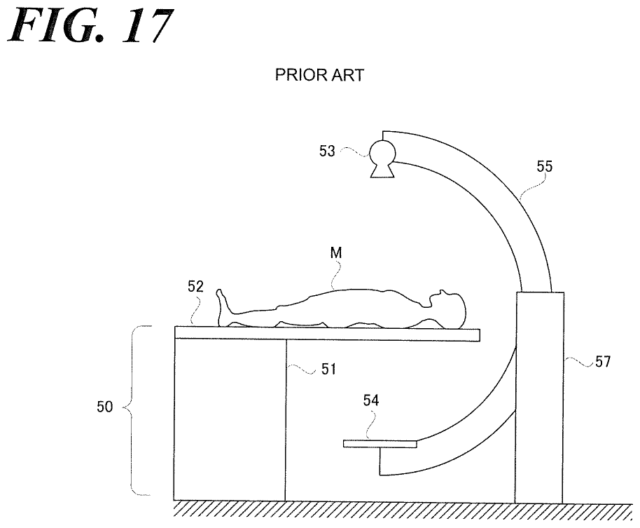

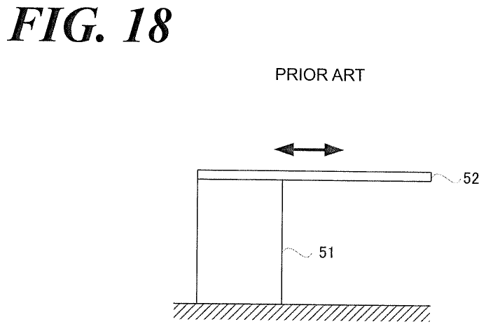

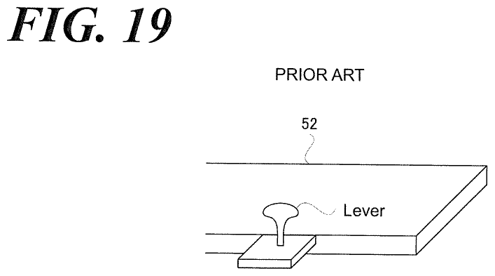

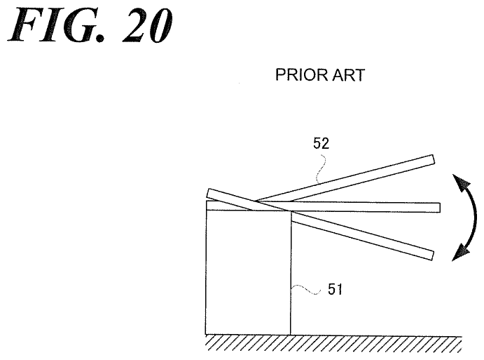

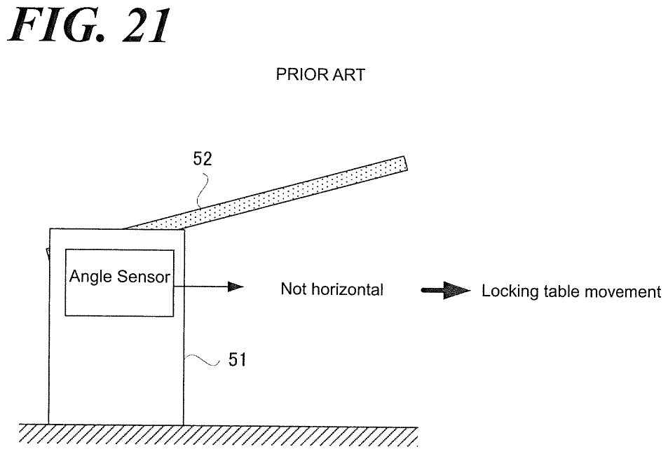

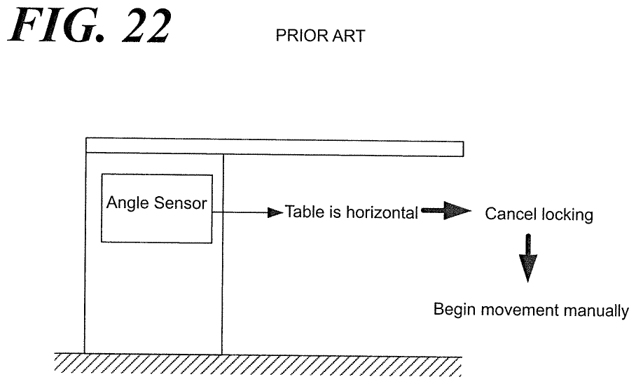

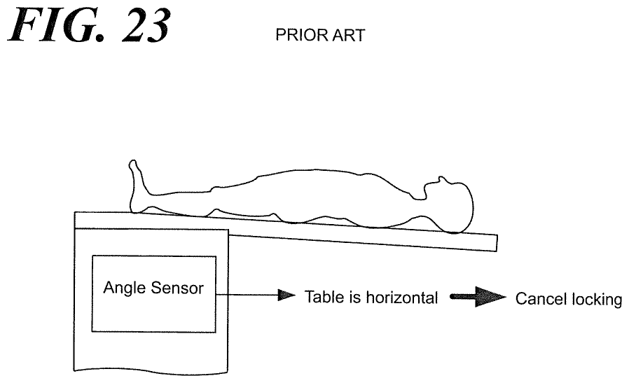

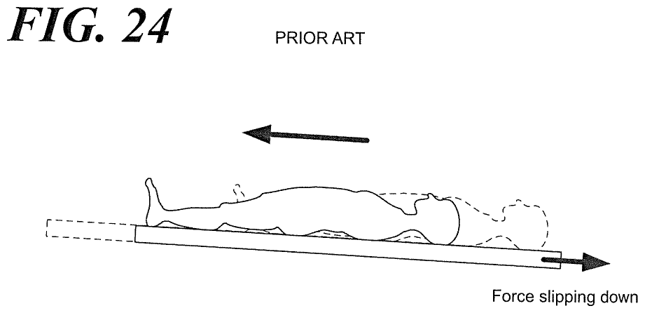

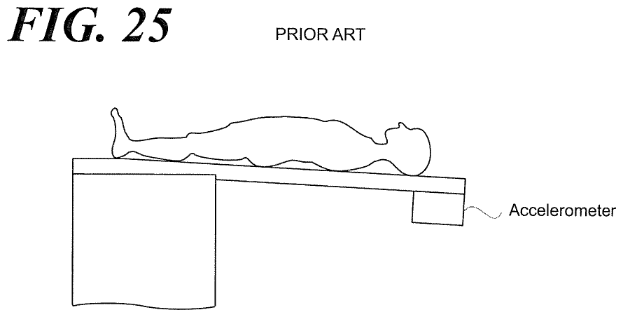

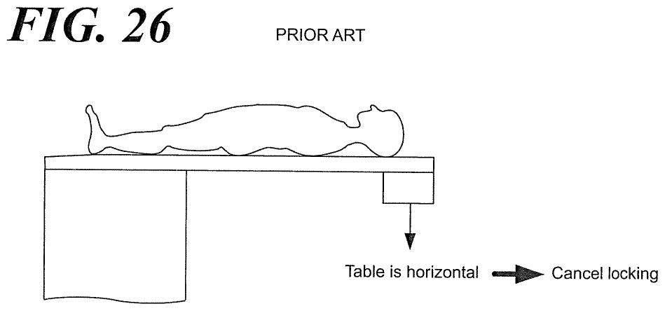

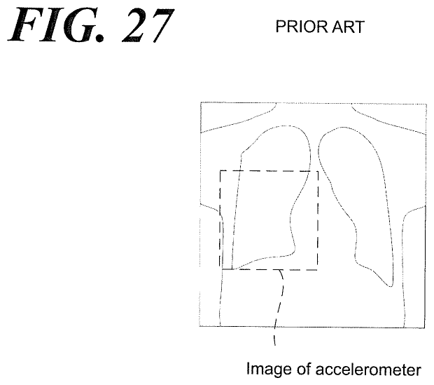

FIG. 8 is a functional block diagram illustrating the horizontal resetting according to the aspect of Embodiment 1.

FIG. 9 is a functional block diagram illustrating unlocking of the table following the angle adjustment according to the aspect of Embodiment 1.

FIGS. 10A, 10B are schematic views illustrating unlocking of the table following the angle adjustment according to the aspect of Embodiment 1.

FIG. 11 is a functional block diagram illustrating unlocking of the table following the angle adjustment according to the aspect of Embodiment 1.

FIGS. 12A, 12B are schematic views illustrating unlocking of the table following the angle adjustment according to the aspect of Embodiment 1.

FIGS. 13A, 13B, 13C, 13D are schematic views illustrating an actual operation of the examination table according to the aspect of the Embodiment 1.

FIG. 14 is a schematic view illustrating an alternative Embodiment of the present invention.

FIGS. 15A, 15B, 15C are schematic views illustrating an alternative Embodiment of the present invention.

FIG. 16 is a schematic view illustrating an alternative Embodiment of the present invention.

FIG. 17 is a schematic diagram illustrating a conventional examination table and a system therefor.

FIG. 18 is a schematic diagram illustrating a conventional examination table.

FIG. 19 is a schematic diagram illustrating a conventional examination table.

FIG. 20 is a schematic diagram illustrating a conventional examination table.

FIG. 21 is a schematic diagram illustrating a conventional examination table.

FIG. 22 is a schematic diagram illustrating a conventional examination table.

FIG. 23 is a schematic diagram illustrating a conventional examination table.

FIG. 24 is a schematic diagram illustrating a conventional examination table.

FIG. 25 is a schematic diagram illustrating a conventional examination table.

FIG. 26 is a schematic diagram illustrating a conventional examination table.

FIG. 27 is a schematic diagram illustrating a problem due to a conventional examination table.

### DETAILED DESCRIPTION OF THE PREFERRED EMBODIMENTS

Reference will now be made in detail to embodiments of the invention. Wherever possible, same or similar reference numerals are used in the drawings and the description to refer to the same or like parts or steps. The drawings are in simplified form and are not to precise scale. The word ‘couple’ and similar terms do not necessarily denote direct and immediate connections, but also include connections through intermediate elements or devices. For purposes of convenience and clarity only, directional (up/down, etc.) or motional (forward/back, etc.) terms may be used with respect to the drawings. These and similar directional terms should not be construed to limit the scope in any manner. It will also be understood that other embodiments may be utilized without departing from the scope of the present invention, and that the detailed description is not to be taken in a limiting sense, and that elements may be differently positioned, or otherwise noted as in the appended claims without requirements of the written description being required thereto.

Various operations may be described as multiple discrete operations in turn, in a manner that may be helpful in understanding embodiments of the present invention; however, the order of description should not be construed to imply that these operations are order dependent.

Hereinafter, the inventor sets forth the best mode of the Embodiment of the present invention. The examination table of the present invention is the examination table having the table on which the subject that is an imaging target of X-ray imaging is loaded. With regard to the X-ray imaging, an X-ray is irradiated toward the subject and the X-ray transmitting through the subject is detected. Such an X-ray passes through not only the subject but also the table on which the subject is loaded. Accordingly, as a given, the X-ray passes through the table of the examination table upon X-ray imaging. The information processing device according to the aspect of the present invention is applied to a control device for an examination table.

### Embodiment 1

Referring to FIG. 1, an examination table of the present invention comprises a pedestal 1 installed on the floor of an examination room and a table 2 supported by the pedestal 1. The table 2 has a structure to load a subject M and is a long-board fitting a figure of the subject M. The longitudinal direction of the table 2 is the body-axis direction A of the subject M to be loaded thereon and the width direction thereof is the side direction S of the body (body-side direction) of the subject M to be loaded thereon. The table 2 has the structure on which the subject M is loaded, and X-ray passes though the structure upon imaging. When imaging the subject M, the acquired image incorporates the table 2 as well. The table 2 is designed as the X-ray passes through, so that the image of the table 2 in the X-ray image does not disturb particularly upon a diagnosis.

The table rotation mechanism 11 that is a movement device is embedded inside the pedestal 1 and rotates the table 2 around the rotation axis parallel to the body-side direction S referring to FIG. 1 relative to the pedestal 1. The table rotation mechanism 11 rotates the table 2 in one direction as the right-edge of the table 2, referring to FIG. 1, rises and also, rotates the table 2 in the reverse direction thereof as the right-edge of the table 2 lowers. The table rotation control element 12 is equipped to control the table rotation mechanism 11. The table rotation mechanism 11 corresponds to the rotation mechanism of the present invention and the table rotation control circuit 12 corresponds to the rotation control mechanism of the present invention.

The table rotation mechanism 11 is equipped with a potentiometer 13. The potentiometer 13 monitors the action of the table rotation mechanism 11 and outputs a signal denoting the gradient-angle of the table 2. A specific method monitors the action of the table rotation mechanism 11 is e.g., the method that measures the rotation amount of the rotation shaft of the table rotation mechanism 11. The output of the potentiometer 13 is sent out to a table gradient-angle calculation circuit 14. The table gradient-angle calculation circuit 14 calculates the gradient-angle of the table 2 based on the output of the potentiometer 13. The potentiometer 13 corresponds to the tilt sensor of the present invention. The potentiometer 13 corresponds to an angle sensor as well.

The operator enables input of the directive relative to the rotation of the table 2 through the table console 2a attached to the side-area of the table 2. The table rotation control circuit 12 controls the table rotation mechanism 11 so that the table 2 rotates in the directed direction and as much as the directed rotation amount based on the directive of the operator through the table console 2a. When The operator operates the table rotation button (switch) attached to the table console 2a, the table rotation control circuit 12 controls the table rotation mechanism 11 so that the table 2 rotates in the directed direction and as much as the directed rotation amount based on the directive of the operator. Consequently, the table 2 rotates around the rotation axis set forth referring to FIG. 1. The table console 2a corresponds to the input device of the present invention.

The table 2 becomes parallel to the floor surface of the examination room at some point while the table 2 is being rotated. When the table is horizontal, the potentiometer 13 outputs the signal denoting such a state. Given the output signal from the potentiometer 13 is e.g., a voltage signal, the output of the potentiometer 13 is a specific voltage (e.g., 100 mV in FIG. 1) when the table 2 is horizontal. When the table 2 that is horizontal rotates in one direction, the output voltage of the potentiometer 13 is getting gradually e.g., smaller from 100 mV. When the table 2 that is horizontal rotates in the reverse direction, the output voltage of the potentiometer 13 is getting gradually e.g., larger from 100 mV. The relationship between the output of the potentiometer 13 and the gradient-angle of the table 2 can be one to one and the gradient-angle of the table 2 is 0° when the output of the potentiometer 13 is 100 mV.

The table gradient-angle calculation circuit 14 actually calculates the gradient-angle of the table 2. The table gradient-angle calculation circuit 14 outputs a signal that indicates that the gradient-angle is 0° when the output voltage of the the potentiometer 13 is 100 mV. In addition, the table gradient-angle calculation circuit 14 outputs the signal that indicates that the gradient-angle is e.g., +1° when the output voltage of the potentiometer 13 is 90 mV and the signal that indicates that the gradient-angle is e.g., −1° when the output voltage of the potentiometer 13 is 110 mV. The output voltage of the potentiometer 13 is called a reference-output when the gradient-angle is 0°. The table gradient-angle calculation circuit 14 stores the setting value of the reference-output and recognizes that the gradient-angle of the table 2 is 0° (horizontal) when the output voltage of the potentiometer 13 coincides with the setting value of the reference-output. The setting value of the reference-output is set up by an expert (maintenance personnel) who adjusts the apparatus by putting on the level on the table 2 to check the horizontal of the table 2. The table gradient-angle calculation circuit 14 achieves the above tasks using a software for controlling the entire table 2. Accordingly, the table gradient-angle calculation circuit 14 should be inherently included in the main control unit 19, but illustrated separately for convenience of understanding the present invention in FIG. 1.

The potentiometer 13 outputs a signal denoting the tilt state of the table 2 to the table gradient-angle calculation circuit 14 by monitoring the action of the table rotation mechanism 11. The table gradient-angle calculation circuit 14 calculates the gradient-angle of the table 2 based on the signal output from the potentiometer 13.

The table console 2a equips with the lever 2a1 that the operator grips. The operator manually slides the table 2 in the body-axis direction A using the lever 2a1 as a grip. Regardless, the table 2 does not automatically slide always as long as the operator operates only the lever 2a1. The reason is that the table 2 rotates around the rotation axis as set forth referring to FIG. 1.

It is so dangerous that the table 2 automatically slips down when the table 2 is slidable under the condition in which the table 2 is tilting. Therefore, the examination table is equipped with the table movement locking mechanism 15 considering such a situation. Subsequently, the operator is unable to manually move the table 2 in the body-axis direction A when the table movement locking mechanism 15 locks the table 2. The table movement locking circuit 16 controls the table movement locking mechanism 15 and provides the table movement locking mechanism 15 with the directive to lock-and-cancel-locking the table 2. In addition, the locking of the table 2 is related to sliding and even locked, the table 2 is rotatable through the table rotation mechanism 11. The table movement locking mechanism 15 corresponds to the movement locking mechanism of the present invention and the table movement control circuit 16 corresponds to the movement locking control mechanism of the present invention. The pedestal 1 supports the table so that the operator manually enables the movement of the table 2 along the body-axis direction A of the subject M.

According to the aspect of the present invention, once the table 2 rotates, the movement locking of the table 2 is activated. When the operator provides the table 2 with the direction relative to rotation while the movement locking of the table 2 is inactivated, the table rotation control circuit 12 sends out the signal, indicating that the table 2 begins rotation from now, to the table movement locking circuit 16. The table movement locking circuit 16 activates the movement locking of the table 2 following receiving such a signal and sends out the signal, indicating that the locking is activated, to the table movement locking circuit 16. The table rotation control circuit 12 carries out rotation of the table 2 following sending the reply signal. Given the operator rotates the table 2 according to such an action, an incident does not take place, in which the table 2 slips down. In addition, when the operator provides the table with the direction relative to rotation while the movement locking of the table 2 is activated, rotation of the table 2 is executed while the movement locking is maintained as an activated condition.

The movement locking mechanism 15 of the present invention activates movement locking of the table 2 to prevent the movement thereof in the body-axis direction. Then, the table movement locking circuit 16 inactivates the movement locking of the table 2 in accordance with the directive of the operator through the table console 2a when the table 2 is horizontal. The table movement locking circuit 16 determines whether the table 2 is horizontal or not based on the output from the table gradient-angle calculation circuit 14.

The lever 2a1 attached to the table console 2a is equipped with a locking cancel button that the operator presses down when canceling locking table-sliding. Specifically, the operator enables movement of the unmovable table 2 using such a locking cancel button. However, it is not guaranteed that the operator always presses down the unlocking button under the condition in which the table 2 is horizontal. Therefore, the examination table is designed as locking of the table 2 is canceled unless the table is horizontal considering such a situation.

FIG. 2 is illustrating the case in which the operator presses down the locking cancel button under the condition in which the table 2 is horizontal. In such a state, the table movement locking control circuit 16 cancels locking following checking that the table 2 is horizontal. Subsequently, the operator enables manually the movement of the table 2 in the body-axis direction A of the subject.

FIG. 3 is illustrating the case in which the operator presses down the locking cancel button under the condition in which the table 2 is not horizontal. In such a state, the table movement locking control circuit 16 cannot confirm that the table 2 is horizontal, so that canceling locking is not carried out. Therefore, it is prevented that the table 2 automatically slips down.

The table movement locking circuit 16 recognizes the gradient-angle of the table 2 based on the output from the table gradient-angle calculation circuit 14. FIG. 2 is illustrating the action of the table movement locking circuit 16 when the output of the table gradient-angle calculation circuit 14 denotes 0° and FIG. 3 is illustrating the action of the table movement locking circuit 16 when the output of the table gradient-angle calculation circuit 14 denotes other than 0°.

FIG. 4 is illustrating that locking of the table 2 is being canceled. When the table is horizontal, the potentiometer 13 outputs the voltage signal of 100 mV. 100 mV implies that the gradient-angle of the table 2 is 0°, so that the table gradient-angle calculation circuit 14 sends out the signal indicating that the gradient-angle of the table 2 is 0° to the table movement locking circuit 16. In such a situation, when the operator presses down the locking cancel button, the table movement locking circuit 16 provides the table movement locking mechanism 15 with the directive for canceling locking. The potentiometer 13 outputs the voltage signal of 100 mV, so that the operator enables canceling locking. When the potentiometer 13 outputs the voltage of 100 mV implying that the table 2 is horizontal upon being provided with the directive for canceling locking by the operator through the table console 2a, the table movement locking circuit 16 cancels the movement locking of the table 2, and reversely when the output voltage of the potentiometer 13 is not 100 mV, locking of the movement is not canceled even when the directive for canceling locking is provided by the operator.

However, when the body weight of the subject M loaded on the table 2 is heavy, the table 2 bends even slightly. Such bending causes tilting the table. As set forth referring to FIG. 24, the gradient of the table 2 makes the operator more difficult to manually slide the table 2 in the body-axis direction A.

The potentiometer 13 cannot detect the gradient of the table 2 due to the bending. FIG. 5A, FIG. 5B are illustrating the rationale therefor. FIG. 5A is illustrating the table 2 that is horizontal without loading the subject M. In such a state, the output of the potentiometer 13 is 100 mV, so that the table movement locking circuit 16 accepts the directive for canceling locking from the operator and cancels locking of the table 2.

It is given that the heavy subject M is loaded on the table 2 as illustrated in FIG. 5A. In such a state, the table 2 bends and consequently, the table 2 slightly tilts as illustrated in FIG. 5B. The potentiometer 13 monitors the action of the table rotation mechanism 11. The table rotation mechanism 11 does not move at all despite loading the subject M on the table 2, so that the output of the potentiometer 13 remains as 100 mV. Then the table movement locking circuit 16 accepts the directive for canceling locking from the operator regardless of the state. The bottom line is that locking of the table 2 is canceled while tilting, so that the incident as illustrated referring to FIG. 24 takes place.

Characteristic Aspects of the Present Invention

The following features are included in the present invention to correspond to such a incident. Specifically, according to the aspect of the present invention, when the operator realizes that the movement of the table 2 is inappropriate, the operator cancels the gradient (tilting) of the table 2. In addition, no accelerometer is mandatory to be mounted on the table 2 differently from the conventional structure, so that an accelerometer never disturbs the X-ray imaging.

FIG. 6 is illustrating that when locking of the table 2 is canceled in the state in which the table 2 is tilting due to bending and the operator realizes hard to manually to move the table 2, the operator operates the way how. Specifically, the operator that realizes that the table is heavy adjusts the angle of the table 2. When the table 2 is easily moved following angle-adjustment, the setting of the table gradient-angle calculation circuit 14 that specifies the horizontal of the table 2 is changed and the updated gradient state of the table 2 is re-specified as a horizontal. Once such an action is carried out, the operator never feels that movement of the table 2 is hard. Referring to FIG. 7, the angle adjustment of the table 2 is carried out by that the operator actually rotates the table 2. Once the table 2 rotates, the movement locking of the table 2 is activated.

When the operator recognizes that the table 2 is getting horizontal while rotating, the operator presses down the horizontal resetting button that is attached to the table console 2a. Then, the definition of horizontal is updated relative to the output of the potentiometer 13. FIG. 8 is illustrating such an operation. When the operator presses down the horizontal resetting button, the signal emerged thereby is output to the horizontal resetting circuit 17. Once the horizontal resetting circuit 17 receives the directive relative to the horizontal resetting from the operator, the horizontal resetting circuit 17 sends out the resetting signal to the table gradient-angle calculation circuit 14. The table gradient-angle calculation circuit 14 sends out the resetting signal, and then reads out the current output value (current value) of the potentiometer and relates the current value to the horizontal state of the table 2. The horizontal resetting circuit 17 corrects an relationship between an output of the potentiometer 13 and a gradient-angle of the table 2 so that the output voltage of the potentiometer 13 denotes that the table 2 is horizontal upon provided with the directive when the operator provides the directive of the correction-execution through the table console 2a and corresponds to the correction circuit of the present invention. As the actual action of the resetting, the main control unit overwrites the data indicating the relationship between the output of the potentiometer 13 and the gradient-angle of the table 2 that the memory storage element 20. Accordingly, the horizontal resetting circuit 17 should be inherently included in the main control unit 19, but illustrated separately for convenience of understanding the present invention in FIG. 1.

Accordingly, the table gradient-angle calculation circuit 14 outputs the signal by which the output of the potentiometer 13 implies that the gradient-angle of the table 2 is 0°. In such a case, the output voltage of the potentiometer 13 is not limited to 100 mV. Referring to FIG. 8, given the output voltage of the potentiometer 13 is 90 mV following adjustment of the gradient-angle of the table 2, the table gradient-angle calculation circuit 14 resets the voltage corresponding to the horizontal position of the table 2 as the output voltage of 90 mV of the potentiometer implies that the table 2 is horizontal following pressing down the horizontal resetting button. Despite the setting value of 100 mV relative to the reference-output, which is stored in the memory storing element 20, the table gradient-angle calculation circuit 14 does not follow such a setting value and is operative as if following the setting value of 90 mV relative to the reference-output, which reflects the adjustment result by the operator. The memory storage element 20 corresponds to the memory circuit of the present invention.

When the setting value of the output voltage (setting value of the reference-output) of the potentiometer 13 relative to the table 2 in the horizontal state is corrected, the output voltage of the potentiometer 13 corresponding to the other gradient-angles of the table 2 varies as well. For example, the table gradient-angle calculation circuit 14 outputs the signal that indicates that the gradient-angle is 0° when the output voltage of the the potentiometer 13 is 100 mV before adjustment and the signal that indicates that the gradient-angle is e.g., −1° following the adjustment. In addition, when the output voltage of the potentiometer 13 is 80 mV, the signal that indicates that the gradient-angle is +1° is output. The examination table according to the present invention includes a function denoting that the table 2 tilts in which direction and how much given the output voltage of the potentiometer 13 is shifted how much from the setting value of the reference-output, so that the relationship between the gradient-angle of the table 2 relative to the other output voltage is also corrected only by correcting the setting value of the reference-output.

In addition, according to the aspect of the present invention, a safety measures for erroneous operation of the operator can be installed. Specifically, given the output voltage of the potentiometer 13 is out of the predetermined range (e.g., 80 mV to 120 mv) having the voltage (100 mV) as the center thereof corresponding to the gradient-angle of 0°, the resetting circuit 17 displays a warning indicating that the table 2 is tilting too much on the table console 2a without correcting the relationship between the output of the potentiometer 13 and the gradient-angle of table 2. According to such an aspect, even when the operator erroneously presses down the horizontal resetting button while the gradient-angle of the table 2 is very large, the movement locking of the table 2 is never canceled. The degree of the bending of the table 2 due to the body weight of the subject is not so large. Accordingly, given the output voltage of the potentiometer 13 is very far from 100 mV, the gradient-angle of the table 2 is very large and too far from the horizontal and in case of canceling the movement locking of the table 2 under such a state, the table 2 likely begins slipping. When the safety measures are installed to the resetting circuit 17, the structure of the aspect of the present invention is much safer.

According to the above explanation, it seems as if the operator easily and accurately recognizes that the table 2 is horizontal by observing the table 2. However, in fact, it is too difficult to know that the table is horizontal by the naked eye. In an actual angle adjustment operation of the table 2, the operator should repeat, in case, four actions including rotation operation of the table 2 through the table console 2a, pressing down the horizontal resetting button, canceling the movement locking of the table 2 and manually sliding the table 2 until the movement of the table 2 becomes very easy and smooth.

Action Following Angle Adjustment

FIG. 9 is illustrating canceling of locking of the table following the angle adjustment of the table 2 according to the aspect of Embodiment 2. When the table is horizontal, the potentiometer 13 in the state in which the table 2 is horizontal, which is newly designated by the operator, outputs the voltage signal of 90 mV. 90 mV implies that the gradient-angle of the table 2 is 0°, so that the table gradient-angle calculation circuit 14 sends out the signal indicating that the gradient-angle of the table 2 is 0° to the table movement locking circuit 16. In such a situation, when the operator presses down the unlocking button, the table movement locking circuit 16 provides the table movement locking mechanism 15 with the directive for unlocking. The potentiometer 13 outputs the voltage signal of 90 mV, so that the operator enables unlocking.

FIGS. 10A, 10B are illustrating the case in which the operator presses down the unlocking button under the condition in which the table 2 is horizontal. In such a state, the table movement locking control circuit 16 cancels locking following checking that the table 2 is horizontal. Subsequently, the operator enables manually the movement of the table 2 in the body-axis direction A of the subject.

FIG. 11 is illustrating canceling locking of the table 2 following the angle adjustment. When the table is horizontal, the potentiometer 13 outputs the voltage signal of 100 mV. 100 mV implies that the gradient-angle of the table 2 is −1°, so that the table gradient-angle calculation circuit 14 sends out the signal indicating that the gradient-angle of the table 2 is −1° to the table movement locking circuit 16. In such a situation, even when the operator presses down the locking cancel button, the table movement locking circuit 16 does not provide the table movement locking mechanism 15 with the directive for canceling locking. The potentiometer 13 outputs the voltage signal of 90 mV, so that the operator enables unlocking.

FIGS. 12A, 12B are illustrating the case in which the operator presses down the locking cancel button under the condition in which the table 2 is not horizontal. In such a state, the table movement locking control circuit 16 cannot confirm that the table 2 is horizontal, so that canceling of locking is not carried out. Therefore, it is prevented that the table 2 automatically slips down and the operator never feels hard when sliding the table 2.

Actual Action of the Examination Table

Finally, the inventor sets forth an actual action of the examination table. When, the operator ends the horizontal resetting operation following adjustment of the gradient-angle of the table 2 referring to FIG. 13A, the definition of the horizontal of the table 2 is changed referring to FIG. 13B. At the time referring to FIG. 13B, the table 2 does not rotate, so that the potentiometer 13 outputs voltage denoting that the table 2 is horizontal. In such a state, when the operator just carries out an operation, the locking of slide of the table is cancelable.

Once the operator provides the table 2 with the directive for rotation through the table console 2a, the table 2 rotates and, referring to FIG. 13C, the table 2 tilts. Once the table 2 begins rotating, the movement locking of the table 2 is activated automatically. Then, it is given that the operator wants to return the table 2 horizontal. In such a situation, the examination table comprises a convenient function. Specifically, the table console 2a equips with a table horizontal button. When the table horizontal button is pressed down, the tilting table 2 automatically rotates to be horizontal as illustrated referring to FIG. 13D.

Such an automatic rotation of the table 2 is achieved by that the table rotation control circuit 12 controls the table rotation mechanism 11 so that the table 2 rotates until the table 2 is horizontal while monitoring the output of the table gradient-angle calculation circuit 14. The horizontal of the table 2 is defined as the state illustrated referring to FIG. 13A, so that the table rotation control circuit 12 rotates the table 2, which is tilting as indicated referring to FIG. 13B, until the table 2 is in the state referring to FIG. 13A. Accordingly, the gradient of the table 2 referring to FIG. 13D is the same as the gradient of table 2 referring to FIG. 13A. Once the table horizontal button is pressed down, the table 2 rotates until the table 2 is into the horizontal state that is reset up. At the time referring to FIG. 13D, the potentiometer 13 outputs voltage denoting that the table 2 is horizontal. In such a state, when the operator just carries out an operation, the locking of slide of the table is cancelable. The table rotation control circuit 12 controls the table rotation mechanism 11 in accordance with the output from the potentiometer 13 so that the table 2 is horizontal.

The initializing circuit 18 cancels correction of the relationship between the output of the potentiometer 13 and the gradient-angle of the table 2, which the horizontal resetting circuit 17 carries out following the end of the radiation imaging of the subject, and then initializes such a relationship. The degree of bending of the table 2 varies depending on the body weight of the subject. Accordingly, even if it is effective with regard to that the correction that is executed by the horizontal resetting circuit 17 carries out smoothly the movement of the table 2 when imaging the subject following adjustment of the angle, the same effect is not guaranteed on the other subject. When such a correction by the horizontal resetting circuit 17 is carried out every imaging, the movement of the table 2 is absolutely and smoothly achieved. Considering such circumstances, the initializing circuit 18 refreshes the relationship between the output of the potentiometer 13 and the gradient-angle of the table 2, every time when the subject loaded on the table 2 changes, to the state before correction executed by the horizontal resetting circuit 17. The initializing circuit 18 initializes the relationship between the corrected output of the potentiometer 13 and the gradient-angle of the table 2 to the default state before correction. The initializing circuit 18 can initialize the relationship when the operator turns off the power of the examination table or can initialize the relationship according to the directive for default through the table console 2a. As the actual action of the initialization, the main control unit overwrites the data indicating the relationship between the output of the potentiometer 13 and the gradient-angle of the table that the memory storage element 20 stores. Accordingly, the initializing circuit 18 should be inherently included in the main control unit 19, but illustrated separately for convenience of understanding the present invention in FIG. 1.

The main control unit 19 comprises CPU that executes a variety of programs and achieves each circuit 12, 16, 17, 18 to be operative. Such circuits can be separately operative by dividing with an arithmetic processing unit device that run each function.

As set forth above, the structure of the present invention comprises the table movement-locking control circuit 16 that cancels the movement locking of the table 2 even though the table 2 is not horizontal when the operator provides the directive for adjusting the gradient-angle detection through the table console 2a, and the horizontal resetting circuit 17 that corrects the output of the table gradient-angle calculation circuit 14 so that the table 2 is processed as horizontal at the time when the operator provides the directive for the correction-execution through the table console 2a.

According to the aspect of the Embodiment 1, it is not mandatory to actually measure how much the table 2 tilts due to bending, so that the accelerometer with the table 2 is no longer mandatory. In fact, the bending of the table 2 varies depending on the body weight of the subject M and the angle, so that the operator must actually and manually conduct a measurement to know the degree of gradient of the table 2 due to the bending. According to the conventional aspects, considering such circumstances, the table 2 is equipped with the accelerometer, whereby the gradient of the table 2, which is actually measured, is canceled by rotating the table 2, so that the table 2 is maintained as a horizontal position. According to the aspect of the present invention, the situation, in which the operator slides the table 2 while changing the angle of the table 2 and realizes that the table 2 is easily slidable, is registered as that the table 2 is horizontal (flat). In such a way, as long as the operator adjusts directly the table 2 to be horizontal, the operator does not have to know the gradient-angle of the table 2 due to the bending using the equipment. According to the aspect of the present invention, the examination table that allows the operator to slide manually and easily is provided, whereby the table 2 is maintained as a horizontal position without equipping the table 2 with the accelerometer that disturbs the radiation imaging.

In addition, as set forth above, given the table movement locking control circuit 16 cancels only in the case of that the gradient of the table 2 is less than the predetermined degree regardless the directive of the operator, it can be prevented that the locking is canceled even when the table 2 is extremely tilting and the risk, in which the table 2 automatically slips down, can be removed.

The present invention is not limited to the above aspects and the following alternative Embodiment can be implemented.

(1) According to the aspect of the Embodiment 1, the horizontal resetting is carried out by that the operator actually tilts the table 2, but the present invention is not limited to such an aspect. Referring to FIG. 14, the table indicating the relationship between the body weight of the subject M and the correction value can be stored in the memory storage element 20. In addition, such a table can represent that the relationship between the bending-angle that is the angle indicating the degree of the gradient of the table 2 due to the bending of the table 2 and the body weight of the subject M loaded on the table 2.

According to the aspect of the present alternative Embodiment, when the operator feels that sliding of the table 2 is heavy and then presses down the horizontal resetting button attached to the table console 2a, the horizontal resetting circuit 17 requests to the main system of the X-ray imaging apparatus to provide the data related to the body weight of the subject M. The body weight of the subject M is input by the operator prior to the X-ray imaging, so that the main system of the X-ray imaging apparatus recognizes the body weight of the subject M. Accordingly, the X-ray imaging apparatus sends out the information relative to the body weight of the subject M to the horizontal resetting circuit 17. The horizontal resetting circuit 17 that recognizes the body weight of the subject M reads out the correction value corresponding to the body weight of the subject M from the memory storage element 20 and resets the setting of the table gradient-angle calculation circuit 14 to be operative by adding the only read-out correction value to the setting value of the reference-output.

Specifically, the horizontal resetting circuit 17 according to the aspect of the present alternative Embodiment corrects the relationship between the output of the potentiometer 13 and the gradient-angle of the table 2 so that the updated state, in which the table 2 rotates as much as the bending angle in the opposing direction against the bending of the table 2 while the potentiometer 13 is detecting the horizontal state, is treated as a horizontal state by reading out the gradient-angle corresponding to the body weight of the subject M from the memory storage element 20 when the body weight of the subject M is input.

According to the aspect of the alternative Embodiment, given the setting value of the reference-output of the potentiometer 13 is 100 mV, the table gradient-angle calculation circuit 14 sends out the signal, indicating that the gradient-angle of the table 2 is 0° when the output of the potentiometer 13 is 100 mV, to the table movement locking circuit 16 prior to pressing down the resetting button. Following pressing down the resetting button, given the body axis direction of the subject M is 70 Kg, the table gradient-angle calculation circuit 14 sends out the signal, indicating that the gradient-angle of the table 2 is 0° when the output of the potentiometer 13 is 96 mV obtained by adding −4 mV, which is read out from the data-table, to 100 mV, to the table movement locking circuit 16.

The data-table indicating the relationship between the body axis direction of the subject M and the correction is generated by actually sliding the table 2, as set forth according to the aspect of the Embodiment 1. Such a data-table is generated by actually carrying out the horizontal resetting every subject M having a different body weight as set forth referring to FIGS. 7A-8B and then obtaining the output voltage of the potentiometer 13 every body-weight when the horizontal resetting is completed.

According to the second aspect thereof, it is also not mandatory to actually measure the angle to know how much the table 2 tilts due to the bending, so that the accelerometer is no longer required to attach to the table 2. According to the aspect of the present alternative Embodiment, the inventor notices the relationship existing between the bending degree of the table 2 and the body weight of the subject M to be loaded on the table 2. The inventor of the present invention realizes that the degree of the bending of the table 2 is comprehensible if the body weight of the subject M is just known. Specifically, in such a case, the actual measurement of the bending of the table 2 is not mandatory. According to the aspect of the present invention, the examination table that allows the operator to slide manually and easily is provided, whereby the table 2 is maintained as a horizontal position without equipping the table 2 with the accelerometer that disturbs the radiation imaging.

(2) According to the above alternative Embodiment (1), the body weight of the subject is noted, but the position of the subject on the table 2 can be noted to carry out the alternative Embodiment. According to the above alternative Embodiment, the data table indicating the relationship between the position of the subject M on the table 2 and the correction value can be stored in the memory storage element 20. Such a data-table can represent that the relationship between the bending-angle that is the angle indicating the degree of the gradient of the table 2 due to the bending of the table 2 and the body weight and position of the subject M loaded on the table 2.

According to the aspect of the present alternative Embodiment, when the operator feels that sliding of the table 2 is heavy and then presses down the horizontal resetting button attached to the table console 2a, the horizontal resetting circuit 17 requests to the main system of the X-ray imaging apparatus to provide the data related to the body weight of the subject M. The position and body weight of the subject M is input by the operator prior to the X-ray imaging, so that the main system of the X-ray imaging apparatus recognizes the position and body weight of the subject M. Accordingly, the X-ray imaging apparatus sends out the information relative to the position of the subject M to the horizontal resetting circuit 17 according to the request from the X-ray imaging apparatus. The horizontal resetting circuit 17 that recognizes the position of the subject M reads out the correction value corresponding to the body weight of the subject M from the memory storage element 20 and resets the setting of the table gradient-angle calculation circuit 14 to be operative by adding the only read-out correction value to the setting value of the reference-output.

(3) According to the aspect of the Embodiment 1, the horizontal resetting is carried out by that the operator actually tilts the table 2, but the present invention is not limited to such an aspect. When the horizontal resetting button is pressed down, the setting value of the reference-output varies just only within the predetermined amount. Specifically, referring to FIG. 15A, when the output voltage of the potentiometer 13 is specified as 100 mV under the condition in which the table 2 is in the horizontal state and the operator presses down the horizontal resetting button, the output voltage of the potentiometer 13 while the table 2 is in the horizontal state is reset to 90 mV which 10 mV is less than the default of 100 mV referring to FIG. 15B. Given the output voltage of the potentiometer 13 is 90 mV in such a state and the operators just carries out the operation, the locking relative to the slide of the table 2 referring to FIG. 15C is canceled.

Specifically, the horizontal resetting circuit 17 according to the aspect of the present alternative Embodiment corrects the relationship between the output of the potentiometer 13 and the gradient-angle of the table 2 so that the updated state, in which the table 2 rotates as much as the predetermined angle in the opposing direction against the bending of the table 2 while the potentiometer 13 is detecting the horizontal state, is treated as a horizontal state when the operator provides the directive for carrying out the correction through the table console 2a.

According to the third aspect thereof, it is not mandatory to actually measure the angle indicating the gradient degree of the table 2 due to the bending, so that the accelerometer is no longer mandatory to be attached to the table 2. In fact, the level of bending of the table 2 varies depending on the body weight of the subject M, so that the operator must actually manually conduct a measurement to know the degree of gradient of the table 2 due to the bending. According to the conventional aspects, considering such circumstances, the table 2 is equipped with the accelerometer, whereby the gradient of the table 2, which is actually measured, is canceled by rotating the table 2, so that the table 2 is maintained as a horizontal position. On the other hand, according to such a conventional aspect, the problem due to the incorporation of the image of the accelerometer, which is set forth referring to FIG. 27, is not recognized. In contrast, according the above aspect of the present invention, the gradient due to the bending of the table is approximately predicted and then the operator maintains the table as a horizontal state by rotating the table 2 as predicted. According such an aspect, an accuracy of the horizontal level of the table 2 to be maintained is certainly lesser than the case in which the gradient of the bending table 2 is actually measured. Regardless, according to the aspect of the above aspect of the present invention, the larger advantage, in which no image of the accelerometer is incorporated, is attained by incorporating no image of the accelerometer. According to the aspect of the present invention, the examination table that allows the operator to slide manually and easily is provided, whereby the table 2 is maintained as a horizontal position without equipping the table 2 with the accelerometer that disturbs the radiation imaging.

(4) According to the above alternative Embodiment (3), no word is given to the adjustment of the predetermined angle, but every time when the horizontal resetting button is pressed down, the setting value of the reference-output varies just only within the predetermined amount. Specifically, when the output voltage of the potentiometer 13 is specified as 100 mV under the condition in which the table 2 is in the horizontal state and the operator presses down the horizontal resetting button once, the output voltage of the potentiometer 13 while the table 2 is in the horizontal state is reset to 98 mV which 2 mV is less than the default of 100 mV. In such a state, when the operator presses down the horizontal resetting button one more time, the output voltage of the potentiometer 13 while the table 2 is in the horizontal state is reset to 96 mV which 4 mV is less than the default of 100 mV. Then after, every time when the operator presses down the horizontal resetting button, the output voltage of the potentiometer 13 while the table 2 is in the horizontal state is getting less as much as just the predetermined amount (2 mV). Specifically, according to the aspect of the present alternative Embodiment based on the number of pressing down the horizontal resetting button, the definition change of the horizontal state of the table 2 can be carried out in incremental steps. Specifically, the horizontal resetting circuit 17 according to the aspect of the present alternative Embodiment is operative using the angle selected from a plurality of the predetermined angles through the table console 2a.

According to the above aspect, the operator adjusts the angle relative to the correction and looks for the more appropriate gradient-angle for the movement of the table 2.

(5) The information processing device according to the aspect of the present invention can be brought into reality by carrying out the following processings. Specifically, such processings are executed by providing the software (programs) that bring the aspects of the above described Embodiments into reality via a network or a variety of memory media to a system or an apparatus and by that a computer (or CPU or MPU and so forth) in such a system or an apparatus reads out such programs and execute.

### REFERENCE OF SIGNS

1 Pedestal
2 Table
2b Operation panel (input device)
11 Table rotation mechanism (rotation mechanism)
13 Potentiometer (tilt sensor)
12 Table rotation control circuit (rotation control mechanism)
15 Table movement locking circuit (movement locking mechanism)
16 Table movement locking control circuit (movement locking control mechanism)
17 Horizontal-resetting circuit (correction mechanism)
20 Memory storage element (memory storage device)

It will be further understood by those of skill in the art that the apparatus and devices and the elements herein, without limitation, and including the sub components such as operational structures, circuits, communication pathways, and related elements, control elements of all kinds, display circuits and display systems and elements, any necessary driving elements, inputs, sensors, detectors, memory elements, processors and any combinations of these structures etc. as will be understood by those of skill in the art as also being identified as or capable of operating the systems and devices and subcomponents noted herein and structures that accomplish the functions without restrictive language or label requirements since those of skill in the art are well versed in related information processing devices, radiation imaging apparatus, diagnostic devices, computer and operational controls and technologies of radiographic devices and all their sub components, including various circuits and combinations of circuits without departing from the scope and spirit of the present invention.

Those of skill would further appreciate that the various illustrative logical blocks, modules, circuits, and algorithm steps described in connection with the embodiments disclosed herein may be implemented as electronic hardware, computer software running on a specific purpose machine that is programmed to carry out the operations described in this application, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the exemplary embodiments.

Having described at least one of the preferred embodiments of the present invention with reference to the accompanying drawings, it will be apparent to those skills that the invention is not limited to those precise embodiments, and that various modifications and variations can be made in the presently disclosed system without departing from the scope or spirit of the invention. Thus, it is intended that the present disclosure cover modifications and variations of this disclosure provided they come within the scope of the appended claims and their equivalents.

1.-10. (canceled)

11. An examination table system, having an information processing device, comprising:
an X-ray transmissive examination table upon which a subject is loaded during a use;
a pedestal that supports said table that during said use an operator manually moves along a body axis direction of said subject;
an input device into which said operator inputs a directive;
a rotation mechanism that rotates said table around a rotation axis during said use;
a tilt sensor that detects a gradient of said table by monitoring an action of said rotation mechanism;
a rotation control circuit that controls said rotation mechanism; and
a movement locking mechanism that activates a movement locking that disables a movement of said table along a body-axis direction;
wherein said information processing device controls said X-ray transmissive examination table on which said subject that is a target is subjected to a radiation imaging, and
wherein:
(A) said table rotates in accordance with said directive provided by said operator through said input device, and said information processing device further comprises:
(B1) a correction circuit that corrects a relationship between an output of said tilt sensor and a tilt angle of said table so that said output of said tilt sensor denotes that said table is horizontal upon being provided with said directive of a correction-and-execution when said operator provides said directive of said correction-and-execution through said input device; and
(C) a movement locking control circuit that cancels said movement locking of said table when said tilt sensor outputs data denoting that said table is horizontal when said operator provides said directive for canceling said movement locking through said input device.

12. An examination table system, having an information processing device, comprising:
An X-ray transmissive examination table on which a subject is loaded during a use;
a pedestal that supports said table that an operator manually moves along a body axis direction of said subject during said use;
an input device into which said operator inputs a directive;
a rotation mechanism that rotates said table around a rotation axis;
a tilt sensor that detects a gradient of said table during said use by monitoring an action of said rotation mechanism;
a rotation control circuit that controls said rotation mechanism; and
a movement locking mechanism that activates a movement locking that disables a movement of said table along a body-axis direction;
wherein said information processing device controls said X-ray transmissive examination table on which said subject that is a target and is subjected to a radiation imaging; and
said information processing device, further comprises:
(m) a memory storage element that stores data-table denoting a relationship between a bending-angle that is an angle indicating the degree of a gradient of said table due to at least one selected from a group consisting of:
a bending of said table, a position of the subject to be loaded on said table, and a body weight of said subject loaded on said table;
(B2) a correction circuit that corrects a relationship between an output of said tilt sensor and a gradient-angle of said table so that the updated state, in which said table rotates as much as a bending angle in an opposing direction against a bending of said table while said tilt sensor is detecting a horizontal state, is treated as a horizontal state by reading out said gradient-angle corresponding to a position of said subject when a body weight of said subject and said position thereof are input, and
(C) a movement locking control circuit that cancels said movement locking of said table when said tilt sensor outputs data denoting that said table is horizontal when said operator provides said directive for canceling said movement locking through said input device.

13. An examination table system, having an information processing device, comprising:
an X-ray transmissive examination table and on which a subject is loaded during a use;
a pedestal that supports said table that an operator manually moves along a body axis direction of said subject during said use;
an input device into which said operator inputs a directive;
a rotation mechanism that rotates said table around a rotation axis,
a tilt sensor that detects a gradient of said table by monitoring an action of said rotation mechanism;
a rotation control circuit that controls said rotation mechanism, and
a movement locking mechanism that activates a movement locking that disables a movement of said table along a body-axis direction;
wherein said information processing device controls said X-ray transmissive examination table on which said subject that is a target subjected to radiation imaging is loaded, and said information processing device, further comprises:
(B3) a correction circuit that corrects the relationship between an output of said tilt sensor and a gradient-angle of said table so that an updated state, in which said table rotates as much as a predetermined angle in an opposing direction against a bending of said table while said tilt sensor is detecting a horizontal state, is treated as a horizontal state when said operator provides said directive to execute correction through said input device, and
(C) a movement locking control circuit that cancels said movement locking of said table when said tilt sensor outputs data denoting that said table is horizontal when said operator provides said directive for canceling said movement locking through said input device.

14. The information processing device, according to claim 13, wherein:
said correction circuit is operative using a predetermined angle that said operator selects through said input device from a plurality of predetermined angles.

15. The information processing device, according to claim 11, wherein:
wherein said information processing device, further comprises:
an initializing circuit that defaults said relationship between a corrected output of said tilt sensor and said gradient-angle of said table to an initial state.

16. The information processing device, according to claim 11, wherein:
said rotation control circuit controls said rotation mechanism to force said table as a horizontal state in accordance with the output of said tilt sensor.

17. A program, that runs a computer that is operative to each circuit in said information processing device, according to claim 11.

18. An X-ray transmissive examination table, comprising said information processing device according to claim 11.

19. A radiation imaging apparatus, comprising said information processing device, according to claim 11.
